(19) United States
(12) Patent Application Publication
(10) Pub. No.: US 2024/0374367 A1
(43) Pub. Date: Nov. 14, 2024

(54) NONUNIFORM EMBROIDERED SOFT TISSUE IMPLANT STRUCTURE

(71) Applicant: TELA Bio, Inc., Malvern, PA (US)

(72) Inventors: John-Paul ROMANO, Chalfont, PA (US); Skott GREENHALGH, Gladwyne, PA (US); Travis SPEICHER, Newtown Square, PA (US)

(21) Appl. No.: 18/353,858

(22) Filed: Jul. 17, 2023

Related U.S. Application Data
(60) Provisional application No. 63/501,300, filed on May 10, 2023.

(51) Int. Cl. A61F 2/00

(52) U.S. Cl. CPC A61F 2/0063; A61F 2002/0068; A61F 2210/0076

(57) ABSTRACT

Described herein are implants (e.g., medical textiles/biotextiles) that include nonuniform stitching patterns to reinforce and bolster the tensile strength of a substrate base and provide customized compliance characteristic to the implant. In some examples, the nonuniform stitching pattern does not substantially change the compliance of the implant relative to the substrate in directions along a plane of the substrate. In some examples, the nonuniform stitching pattern creates a nonuniform directional compliance characteristic to the implant. The implants may be configured as surgical grafts that may be used for soft tissue reconstruction, regeneration, or repair.

### CLAIM OF PRIORITY

This application claims the benefit of U.S. Provisional Patent Application No. 63/501,300, filed May 10, 2023 and titled “NONUNIFORM EMBROIDERED SOFT TISSUE IMPLANT STRUCTURE,” which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The apparatuses and methods described herein relate generally to surgical grafts and medical textiles useful for soft tissue reconstruction, regeneration, or repair. More particularly, described herein are surgical implants for soft tissue repair that include nonuniform stitching pattern that imparts tensile strength and a customized compliance to the implant.

### BACKGROUND

Soft tissues within a body may benefit from repair or reinforcement due to a variety of reasons such as disease, enhancement, or trauma.

An implant or medical textile may be used to repair or reinforce a soft tissue, such as an unhealthy or modified tissue in the body. The tissue may, for example, be tissue that is no longer able to maintain its shape or physiological function such as a hernia or a tissue for which a shape or size change is desired such as breast size or shape change due to breast enhancement or breast reconstruction. A hernia is a condition in which part of an organ or fatty tissue protrudes through the wall of a surrounding tissue. Abdominal wall hernia surgery is one of the most common surgical procedures, and according to the U.S. Food and Drug Administration, more than 1 million hernia repairs are performed in the United States alone. Common adverse events associated with hernia repair surgery include pain, infection, hernia recurrence, adhesion formation, obstruction, bleeding, and fluid build-up. Breast reconstruction may be performed to reconstruct a breast after a mastectomy has been performed to remove a diseased tissue due to cancer or as a prophylactic measure to prevent cancer. Common adverse events associated with breast reconstruction include infection, pain, delayed healing, and swelling.

There is a need for improved surgical repair materials and medical textiles.

### SUMMARY OF THE DISCLOSURE

Described herein are surgical implants including grafts and medical textiles that include a nonuniform stitching pattern embroidered into a substrate. The substrate may include one or more layers of a biotextile (biologic) or medical textile (polymer based). The nonuniform stitching pattern may provide localized tensile strength to the implant yet allow the implant to deform in multiple directions with respect to a plane of the substrate, or provide the implant with unequal extents of compliance in different directions along the plane of the implant.

The implants may be used in the repair and/or plastic reconstruction of soft tissue. For example, the implants may be implemented as a surgical repair material, surgical repair mesh (or scaffold or patch), a hernia mesh (or scaffold or patch), etc. The surgical repair implant may be useful for supporting or repairing a body tissue such as for breast reconstruction, hernia repair, pelvic organ prolapse treatment, among other soft tissue repair and reconstruction uses.

According to one example, an implant for repairing or reconstructing soft tissue includes: a substrate comprising a biotextile or a medical textile having an upper surface and a lower surface; and a nonuniform stitching pattern embroidered into the substrate that increases a strength of the implant relative to the substrate without changing a compliance of implant relative to the substrate, wherein the nonuniform stitching pattern includes one or more filaments stitched into curved lines forming a plurality of adjacent cells having non-parallel sides across the upper surface of the substrate.

The one or more filaments stitched into curved lines may have a radius of curvature that varies along the curved lines. The absolute value of the radius of curvature may be greater than 3 millimeters.

The nonuniform stitching pattern may be configured so that the one or more filaments intersect at corners of the plurality of adjacent cells at a shared intersection stitching point. The plurality of adjacent cells may have a plurality of different shapes comprising two-sided, three-sided, four-sided, five-sided, and six-sided shapes. The plurality of adjacent cells may form an irregular pattern across the upper surface of the substrate.

The substrate may include a plurality of layers of material. The nonuniform stitching pattern may be stitched into two or more of the plurality of layers of materials. The substrate may include a sheet of extracellular matrix material (ECM). The implant may be configured as a graft for one or more of hernia repair and tissue reconstruction.

The plurality of adjacent cells may have a distribution of surface areas greater than 1 mm². The plurality of adjacent cells may have a distribution of areas between about 1 mm² and 100 mm². The plurality of adjacent cells may have approximately the same area.

The implant may further include a border stitch around a perimeter of the implant.

The one or more filaments may include a biocompatible material. The biocompatible material may be resorbable.

According to another example, an implant for repairing or reconstructing soft tissue includes: a substrate comprising a biotextile or a medical textile having an upper surface and a lower surface; and a nonuniform stitching pattern embroidered into the substrate, wherein the nonuniform stitching pattern includes one or more filaments stitched into curved lines forming a plurality of adjacent cells having non-parallel sides across the upper surface of the substrate, wherein the one or more filaments stitched into curved lines has a radius of curvature that varies along the curved lines, so that the implant has the same compliance in any direction in the plane of the substrate.

According to further example, a method for forming an implant for repairing or reconstructing soft tissue includes: stitching one or more filaments into a substrate to form a nonuniform stitching pattern in the substrate, the substrate comprising a biotextile or a medical textile, where the nonuniform stitching pattern increases a strength of the implant relative to the substrate without changing a compliance of implant relative to the substrate, wherein the nonuniform stitching pattern includes the one or more filaments stitched into curved lines forming a plurality of adjacent cells having non-parallel sides across an upper surface of the substrate, so that the implant has the same compliance in any direction in the plane of the substrate.

The method may further include testing the compliance in one or more directions along the plane of the substrate to confirm that the implant has the same compliance in any direction in the plane of the substrate. The method may further include determining the nonuniform stitching pattern. The substrate may include one or more layers of the biotextile or the medical textile, wherein stitching the one or more filaments into the substrate includes stitching the one or more filaments into the one or more layers of the biotextile or the medical textile. Stitching the one or more filaments into the substrate may include stitching the one or more filaments so that the curved lines have radius of curvature that varies along the curved lines. Stitching the one or more filaments into the substrate may include stitching the one or more filaments in a lock-stitch configuration. Stitching the one or more filaments into the substrate may include stitching one or more non-parallel lines, curved shapes, circles, ovals, irregular shapes, loops, dots, or any combination thereof into the substrate.

According to another example, a method of using an implant for repairing or reconstructing soft tissue includes: surgically implanting the implant with a patient's body, wherein the implant includes a nonuniform stitching pattern embroidered into a substrate that increases a strength of the implant relative to the substrate without changing a compliance of implant relative to the substrate, wherein the nonuniform stitching pattern includes one or more filaments stitched into curved lines forming a plurality of adjacent cells having non-parallel sides across an upper surface of the substrate.

Surgically implanting the implant may include orienting the upper surface of the substrate nearer to a particular tissue type of the patient's body relative to a lower surface of the substrate that is opposite the upper surface.

These and other examples are described further herein.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 illustrates one side (e.g., front side) of an example soft tissue implant having a uniform stitch pattern.

FIG. 2 illustrates one side (e.g., front side) and a close-up view of an example soft tissue implant having a nonuniform stitch pattern.

FIG. 3 illustrates an expanded view of a substrate with multiple layers of material.

FIG. 4 illustrates a side view and a close-up view of an example implant with a lock-stitch configuration.

FIGS. 5A-5H illustrate examples of different nonuniform stitching patterns.

FIG. 6 is a flowchart illustrating an example method of forming a soft tissue implant with a nonuniform stitching pattern.

### DETAILED DESCRIPTION

Described herein are soft tissue implants including textiles (e.g., biotextiles and/or medical textiles) that include a substrate into which a filament (which may also be referred to as a thread, yarn, fiber, suture, or strand) is sewn and formed into a nonuniform stitch pattern. The nonuniform stitching pattern may be used to locally increase the strength of the substrate while controlling a degree of compliance of the substrate. In some variations, the nonuniform stitching pattern does not substantially decrease the overall compliance of the implant relative to the substrate, thus allowing the implant to deform and stretch in multiple directions. In other variations, the nonuniform stitching pattern nonuniformly increases the compliance of the implant such that the implant is more compliant in certain directions than others.

Biotextile or medical textile material are typically used for soft tissue repair or reconstruction and may be surgically implanted within the body. Such soft tissue implants may serve, for example, to replace or reinforce diseased or damaged soft tissue, or to hold internal organs in place in the case of a hernia repair. In some cases, these soft tissue implants are intended to be a permanent fixture within the body, for example, medical textile implants comprising permanent polymeric threads. In other cases, the soft tissue implants are intended to be a temporary fixture within the body such that they are made of a material that is gradually resorbed by the body as it is replaced by the body's own tissue, for example, biotextile implants comprising an extracellular matrix. Regardless, patients do not all heal at the same rate, owing to the particular condition in need of repair, and the physical characteristics and conditions of the patient. Accordingly, it is desirable to control the inherent base properties of such implants to accommodate the conditions of individual patients. In addition, it is desirable to compensate for premature breakdown or resorption of the implant.

The nonuniform stitching pattern may be used to locally increase the strength of a substrate while controlling the degree of compliance of the resulting implant. As used herein, a nonuniform stitching pattern refers to a stitching pattern that has a varied geometric characteristic. For example, a nonuniform stitching pattern may include stitches that form nonuniform shapes, such as curved lines (e.g., wavy lines, looped lines), geometric shapes with curved sides, irregular geometric shapes or lines, complex geometric shapes, or other nonuniform shapes. Alternatively or additionally, a nonuniform stitching pattern may include shapes (uniformly shaped or nonuniformly shaped) that are distributed in a nonuniformly arrangement on the substrate. For example, a nonuniform stitching pattern may include a nonuniform arrangement of lines (e.g., non-parallel lines or irregularly arranged lines) and/or a nonuniform arrangement of geometric shapes (e.g., circles, ovals, dots). A nonuniform stitching pattern may include nonuniform shapes that are nonuniformly arranged on the substrate. In some variations the shapes intersect with each other on the substrate. In other variations the shapes do not intersect with each other on the substrate. In some variations, the nonuniform stitching pattern includes a pattern of smooth lines that do not meet at sharp angles so as to avoid stress concentrators.

In some variations, the nonuniform stitch pattern does not change (or does not substantially change) the overall compliance of the implant relative to the substrate. Compliance refers to the ability of an implant or substrate to or deform or stretch in response to certain load forces and is a function of, among other things, strength, stress, elongation, rebound, deformability, and elasticity properties of the particular materials or combination of materials. Compliance and tensile strength may be measured along a plane of the implant or substrate in one or more directions. In some examples, not substantially changing the compliance means changing the compliance of the substrate by no more than 10% (e.g., by no more than 10%, 8%, 7%, 6%, 5%, 4%, 3%, 2% or 1%). In some variations, the nonuniform stitch pattern may be configured not to substantially change the overall compliance of the implant in multiple directions (e.g., all directions) compared to the substrate without the nonuniform stitch pattern. In other variations, the nonuniform stitch pattern may be configured to change the compliance of the substrate such that the implant is more compliant in one or more predetermined directions along the plane of the substrate compared to the substrate without the nonuniform stitch pattern.

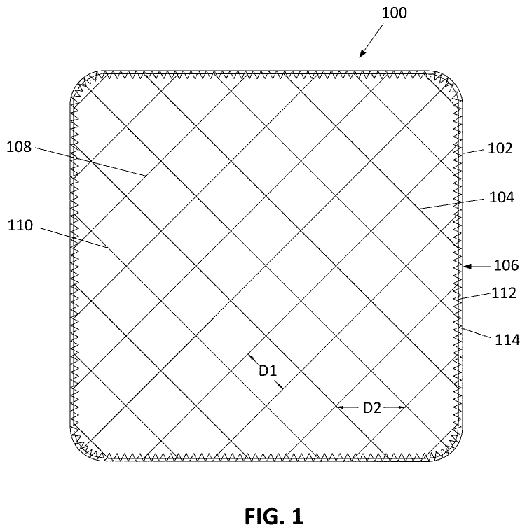

FIG. 1 shows an example implant 100 having a uniform stitching pattern. The implant 100 includes a substrate 102 that includes a filament 104 that is stitched into the substrate in a pattern of parallel lines (crosshatched pattern). The crosshatched pattern includes a first series of parallel stitch lines 108 that intersects with a second series of parallel stitch lines 110. In this example, the crosshatched pattern forms squares having sides of length D1 and diagonal distances D2. Also in this example, the implant 100 includes a border stitch 106, which includes a line stitch 112 that follows the perimeter edge of the substrate 102, and a zig-zag stitch 114 that is stitched over the line stitch 112 in a zig-zag pattern. In addition to strengthening the substrate 102, the uniform stitching pattern may increase the compliance of the implant 100 relative to the substrate 102.

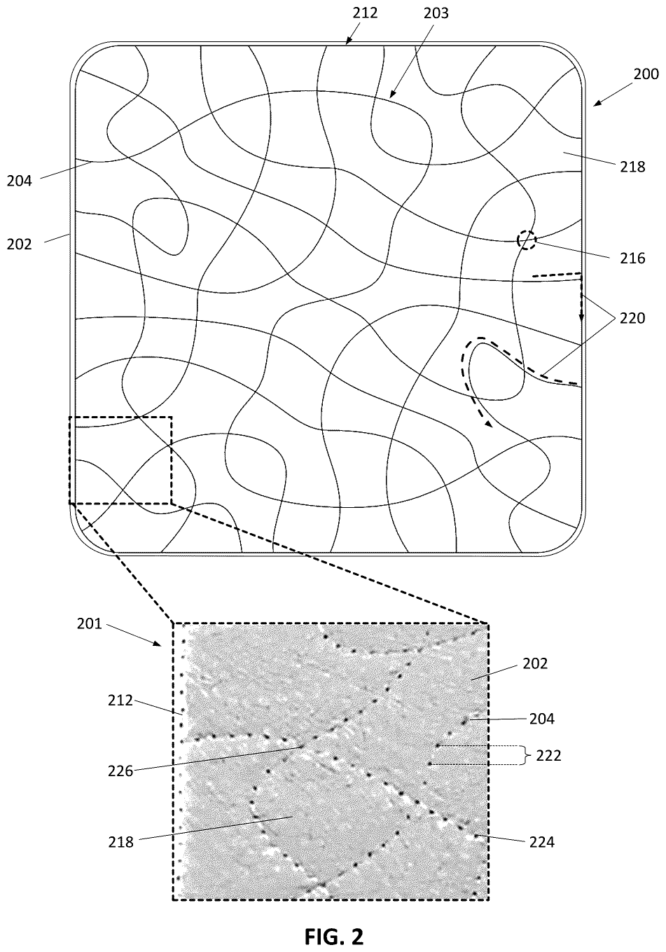

FIG. 2 shows an example soft tissue implant 200 having a nonuniform stitching pattern 203, according to some embodiments. A filament 204 is stitched in a substrate 202 along one or more embroidery paths 220 that form the nonuniform stitching pattern 203 and a border stitch 212. The embroidery path(s) 220 may include curved sections, straight sections, and angled direction changes. In this example, the nonuniform stitching pattern 203 includes a pattern of curved lines that intersect at intersections 216. The curved lines may create a smooth path to avoid stress concentrators. As shown, none of the lines in the nonuniform stitching pattern 203 are parallel to each other. The border stitch 212 follows the perimeter edge of the substrate 202 and surrounds the area of the substrate 202 with the nonuniform stitching pattern 203. In this example, the border stitch 212 includes a single line stitch. In other examples, the border stitch may alternatively or additionally include a different type of stitch, such as a zig-zag stitch similar to the zig-zag stitch 114 in the example of FIG. 1.

The nonuniform stitch pattern 203 can provide localized tensile strength to the implant 200. In this way, the implant 200 can withstand localized tensile forces applied to when implanted within a patient's body. In the aggregate, the nonuniform stitch pattern 203 may not provide a particular directional compliance contribution. For example, the nonuniform stitch pattern 203 may cause the implant 200 not to deform more in any one direction along the plane of the substrate 202. That is, the implant 200 can deform and stretch in multiple directions, and may not be biased to deform/stretch in any one particular direction. In this way, the nonuniform stitch pattern 203 may provide the implant 200 with more overall in-plane compliance (along the plane of the substrate 202) compared to the uniform crosshatched stitch pattern of implant 100.

The nonuniform stitch pattern 203 forms smooth lines that do not meet at sharp angles so as to avoid stress concentrators. In the example shown, the curved lines of the nonuniform stitch pattern 203 have a radius of curvature that varies along the curved lines. In some cases, the absolute value of the radius of curvature of the curved lines is greater than three (3) millimeters (mm). In some variations, the overall radius of curvature of the curved lines may be between one tenth ( 1/10^{th}) and one one-hundredth ( 1/100^{th}) of the overall area of the substrate 202. In some variations, the radius of curvature of the curved lines ranges between one eighth (⅛^{th}) of an inch to one tenth 1/10^{th}) of an inch.

As shown in the close-up view 201 of a portion of the implant 200, the filament 204 penetrates the substrate 202 multiple times to form multiple stitches 222 where the filament 204 is exposed on one side of the implant 200 (side shown in FIG. 2). The nonuniform stitch pattern 203 may be chosen so as to reduce or minimize the exposure of the filament 204 on one side of the implant 200 (e.g., side shown in FIG. 2). This may be advantageous in cases where it is preferable to limit or minimize contact of the material of the filament 204 to certain body tissues (e.g., bowels). For example, some polymer materials in which the filament 204 may be made from a polymer material that may tend to adhere to certain body tissues (e.g., as compared to the material of the substrate 202). However, if the amount of filament 204 is reduced too much, the substrate 202 will not have sufficient tensile strength. Thus, the nonuniform stitch pattern 203 may be chosen based on a balance between providing sufficient tensile strength/compliance as and reducing exposure of the filament 204 to certain tissues (to at least an exposed side of the implant 200). In this particular case, the nonuniform stitch pattern 203 may reduce exposure of the filament 204 on the front side of the implant 200 shown in FIG. 2 by 33.6% compared to exposure of the filament 104 in the crosshatched stitch pattern on the front side of implant 100 shown in FIG. 1.

Also as shown in the close-up view 201, the filament 204 passes through holes 224 in the substrate 202 created during the stitching process. At the intersections 216 of the curved lines, the filament 204 passes through shared holes 226 in the substrate 202. This configuration may help to maintain the integrity (reduce weakening) of the substrate 202 compared to stitching arrangements where the substrate 202 has multiple holes for accommodating the filament 204 at each intersection 216. In some variations, the hole 226 at each of the intersections 216 is the same size (e.g., diameter) as non-shared holes 224, which may also help to maintain the integrity (reduce weakening) of the substrate 202 compared to larger holes to accommodate multiple passes of the filament 204.

The nonuniform stitch pattern 203 can form cells 218, which correspond to areas of the substrate 202 having boundaries defined by the nonuniform stitch pattern 203 of the filament 204. The cells 218 have non-parallel sides in accordance with the non-parallel curved lines of the nonuniform stitch pattern 203. The filament 204 intersects at shared intersection stitching points 216 at corners of the cells 218. As shown, the cells 218 may have different shapes and have a different number of sides (e.g., two-sided, three-sided, four-sided, five-sided, and six-sided shapes). The cells 218 themselves may form a nonuniform pattern across the surface of the substrate 402.

The nonuniform stitch patterns described herein (including the nonuniform stitch pattern 203) may be designed such the size (area) of each of the cells is the same or similar (e.g., differ in area by no more than 1% to 10% (e.g., 1%, 3%, 5%, 8%, or 10%). In some variations, the nonuniform stitch pattern 203 is designed such that the area of the cells varies (e.g., vary in area by more than 10% to 99% (e.g., 10%, 12%, 15%, 20%, 30%, 35%, 40%, 50%, 60%, 70%, 80%, 90%, 95% or 99%). In some variations, whether the area cells are the same or vary, the cells may have a consistent average cell area across the substrate. In some cases, the cells may have a distribution of surface areas greater than 1 mm². In some cases, the cells have a distribution of surface areas between about 1 mm² and 100 mm².

In some variations, the cells of any of the nonuniform stitch patterned implants described herein (such as cells 218 of implant 200) may be configured to absorb fluid to form pockets of fluid within the implant. For example, if the substrate includes multiple layers of material, the fluid may enter the cells between adjacent layers. In some cases, this may advantageously increase the local strength of the implant without impacting the overall compliance of the implant. In some examples, the swelling of cells with fluid may the change the surface contour of the substrate. For example, such swelling of the cells may at least partially eclipse the filament such that less of the filament is exposed on the outer surface implant, which may be desirable in some cases.

Any of a number of suitable materials may be used for the filaments described herein, including polymeric materials and non-polymeric materials. For example, the filament may be a monofilament or include multiple sub-filaments. Examples of filaments polymer materials may include one or more permanent polymers (e.g., polyethylene terephthalate (PET), polypropylene, nylon, polytetrafluoroethylene (PTFE)). Other permanent materials (metals, organic materials, etc.) may additionally or alternatively be used. In some examples, the filament is made of a biodegradable (e.g., resorbable) polymer. In some variations the stitching is made from one or more filaments that include a mixture of permanent and biodegradable (e.g., resorbable) polymers.

The substrate in any of the examples described herein may be made of any of a number of materials. Non-limiting examples of substrate materials may include cloth or fabric, lace, leather, silk, linen, nylon, polyester, polypropylene, polyethylene, cotton, satin, wool, bamboo, cashmere, jute, burlap, fleece, felt, spandex, rayon, denim, and other suitable materials, or any combination thereof. In some cases, the substrate is made of a textile material, which may be obtained or derived from living tissue and/or be a synthetic material. Living tissue may include, for example, dermis/skin tissue, sub-tissue, extracellular matrices (ECMs), pericardium, peritoneum, intestine, stomach, forestomach, and other suitable tissues. The animal source may be any suitable animal, including a human, pig, cow, or sheep, or may be synthesized, for example, by recombinant expression. In some cases, the substrate material may be biodegradable or resorbable. Some non-limiting examples of biotextiles include extracellular matrix-derived tissue scaffolds, autograft tissue, allograft tissue, and xenograft tissue, as well as artificial skin, artificial heart valves, and other implantable prosthetics. Some non-limiting examples of synthetic materials include polypropylene, polyethylene, and/or other implantable polymer materials. The substrate may be woven or non-woven. In some cases, the substrate may be or include a mesh obtained or derived from living tissue and/or made of a synthetic material. Any of the substrates described herein may have a smooth surface, a textured surface, or a combination of smooth and textured surfaces.

Any of the substrates and/or filaments described herein may be coated and/or impregnated with one or more agents (e.g., antibiotics and/or anti-inflammatory agents). Any of the sewn textures may include (including as a filament or part of a filament) a radiopaque material such as, but not limited to, barium doped or metallic (gold, Pt, Pt Iridium, etc.).

Any of the substrates described herein may include any number of layers (also referred to as sheets) of material. For example, a substrate may include one, two, three, four, five, six, seven or more layers of material. If the substrate includes two or more layers of material, the nonuniform stitching pattern may secure two or more of the layers together.

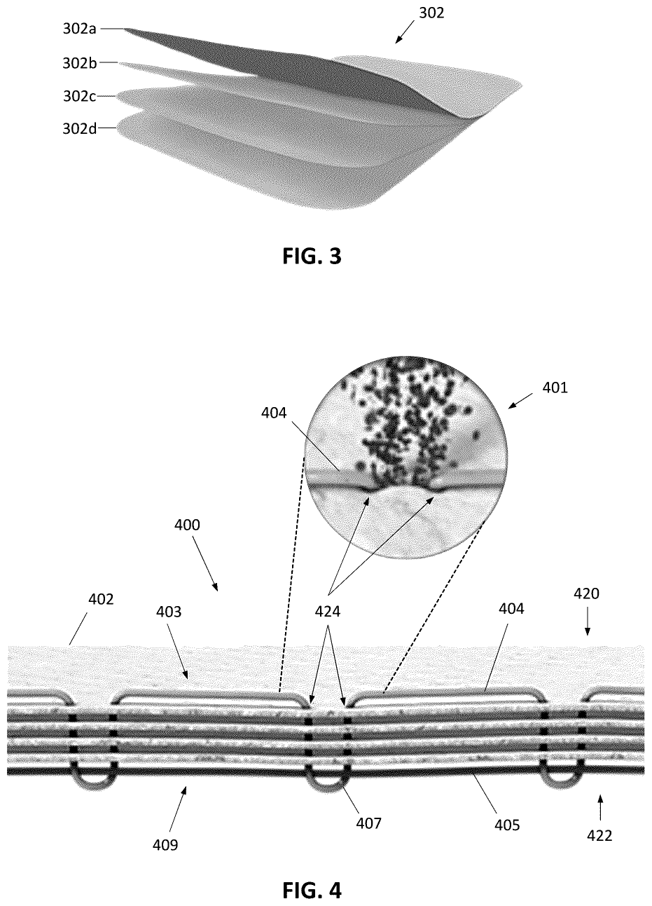

FIG. 3 shows an example substrate 302 that includes four layers 302a, 302b, 302c and 302d of material. The nonuniform stitching pattern may be stitched through one, two, three or all four of the layers 302a, 302b, 302c and 302d. In some variations all layers of a substrate are made of the same material, while in other variations the layers are made of one or more different materials.

The nonuniform stitch patterns described herein may be stitched in a substrate in a lock-stitch configuration/pattern where one side of the implant has a different stitch configuration than the opposite side of the implant. FIG. 4 shows a side view and a close-up view of an example implant 400 illustrating one example of a lock-stitch configuration. In this example, the substrate 402 includes multiple (i.e., four) layers of material. A first (e.g., upper) filament 404 is sewn through the substrate 402 such that a nonuniform stitching pattern 403 is formed on a first (e.g., upper) side 420 of the implant 400 (and substrate 402). As shown, the first filament 404 forms a loop 407 and captures a second (e.g., lower) filament 405 on a second (e.g., lower) side 422 of the implant 400 (and substrate 402), thereby forming the lock-stitch configuration. The loop 407 of the first filament 404 and the second filament 405 form a reverse stitch pattern 409 on the second side 422 of the substrate 402.

In some examples, the first filament 404 and the second filament 405 are separate filaments. The first filament 404 may be made of the same material as the second filament 405, or may be made of a different material than the second filament 405. In other examples, the first filament 404 and the second filament 405 are continuous with each other and part of the same filament.

The lock-stitch configuration results in the first side 420 of the implant 400 generally having a smoother surface contour than the second side 422 of the implant 400. In addition, the first side 420 of the implant 400 generally has less surface area taken by a filament (i.e., filament 404) than the second side 422 of the implant 400. Thus, the first side 420 and second side 422 of the implant 400 may have different surface properties that may dictate which side is preferentially placed next to certain tissues when implanted within a patient's body. For example, it may be preferable to place the smoother first side 420, which includes the nonuniform stitching pattern 403, against tissue areas closer to certain body organs relative to the rougher second side 422. In some variations, the material (e.g., type of polymer) of the first filament 404 may be chosen to minimize biological response with body tissue, which may or may not be different than the material of the second filament 405 that may not be expected to interact with tissue to the same extent as the first filament 404 when implanted in the body.

As discussed previously, the first filament 404 passes through holes 424 of the substrate 402 to create the lock-stitch configuration. As illustrated in the close-up view 401, the holes 424 may provide pathways (channels) for bodily fluid (e.g., blood) to transfer through the substrate 402, which may promote absorption of bodily fluid during implantation of the implant 400 and enable cellular migration during healing.

The nonuniform stitching patterns described herein may form any of a number of various shapes and patterns on the surface of the implant. In some variations, the nonuniform stitching patterns form irregular shapes. In other variations, the nonuniform stitching patterns form uniform or nonuniform shapes that are nonuniformly distributed on the substrate.

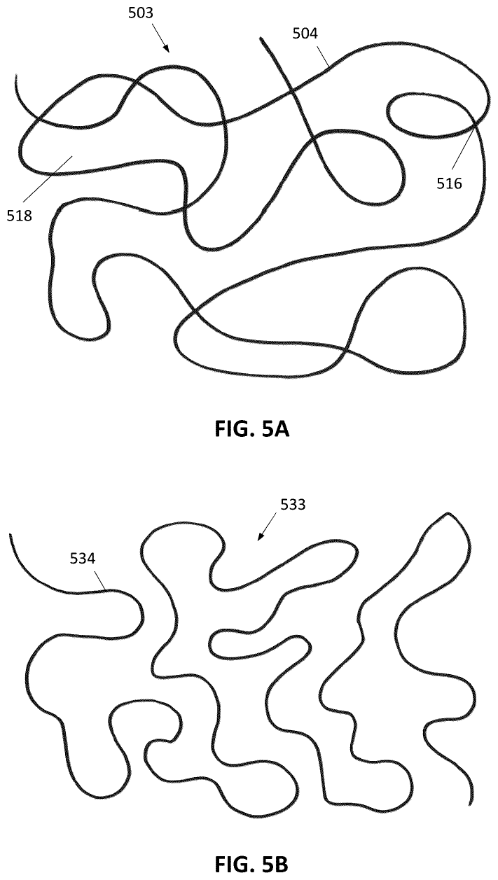

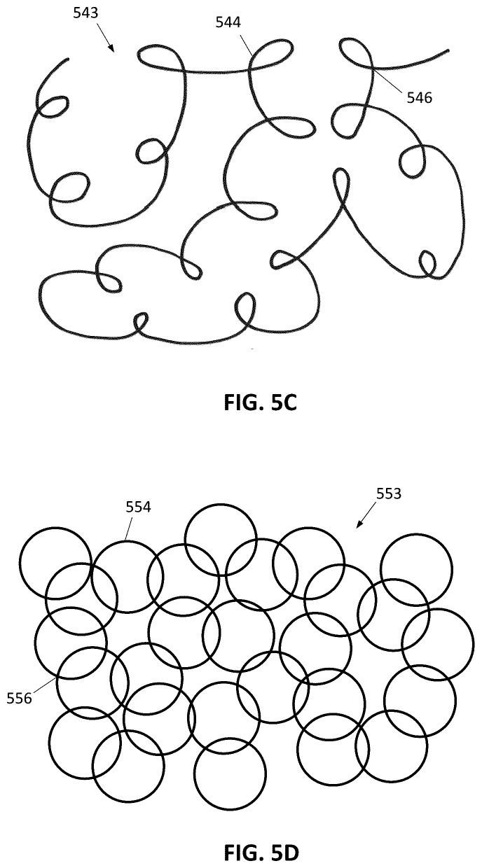

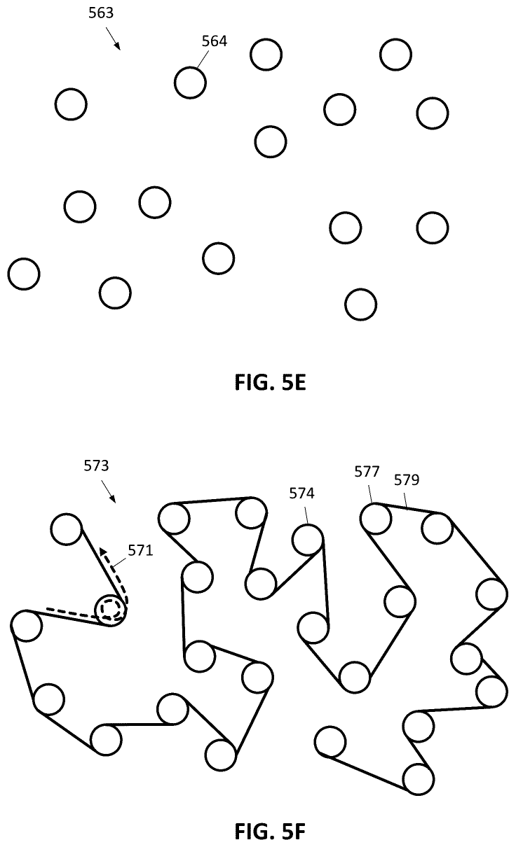

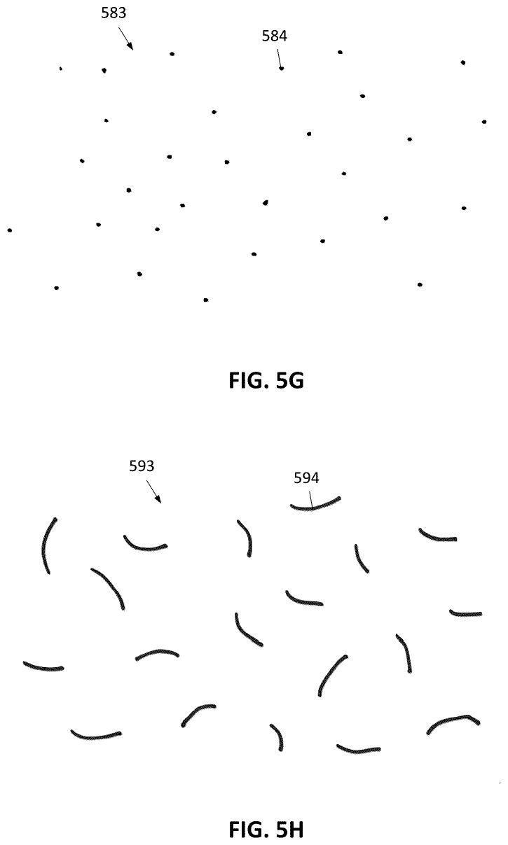

FIGS. 5A-5H show examples of different nonuniform stitching patterns. FIG. 5A shows an example nonuniform stitching pattern 503 in which a filament 504 forms a curved line that crosses itself. The nonuniform stitching pattern 503 is similar to the nonuniform stitching pattern 203 in FIG. 2 but with a different size distribution of cells 518. In some variations, the filament 504 may enter the substrate shared holes at intersection points 516, similar to the hole 226 of the substrate 202 discussed above with reference to FIG. 2.

FIG. 5B shows an example nonuniform stitching pattern 533 in which a filament 534 forms a curved line that does not cross itself.

FIG. 5C shows an example nonuniform stitching pattern 543 in which a filament 544 forms a tightly curved (curly) line that crosses itself. In some variations, the filament 544 may enter the substrate shared holes at intersection points 546, similar to the hole 226 of the substrate 202 discussed above with reference to FIG. 2.

FIG. 5D shows an example nonuniform stitching pattern 553 in which a filament 554 forms a series of circles (also referred to as bubbles) that cross each other. In the example shown, the circles are the same size (diameter). In other examples, the circles may be different sizes (diameters). In some examples, the stitching pattern includes closed curved shapes other than circles, such as ovals, irregularly shaped closed shapes, or a combination thereof. In some variations, the filament 554 may enter the substrate shared holes at intersection points 556, similar to the hole 226 of the substrate 202 discussed above with reference to FIG. 2.

FIG. 5E shows an example nonuniform stitching pattern 563 in which a filament 564 forms a series of circles that do not cross each other, thereby forming a series of islands. The circles may be equidistantly distributed or non-equidistantly distributed on the substrate surface. The circles may be randomly (or pseudo-randomly) distributed on the substrate surface, or distributed in a predetermined pattern on the substrate surface. In the example shown, the circles are the same size (diameter). In other examples, the circles may be different sizes (diameters).

FIG. 5F shows an example nonuniform stitching pattern 573 in which a filament 574 forms a series of circles 577 that are connected to each other by lines 579, thereby forming a series of connected islands. In the example shown, the circles are the same size (diameter). In other examples, the circles may be different sizes (diameters). In some cases, each circle 577 is formed by forming a loop such that the filament 574 overlaps with itself as indicated by arrow 571.

FIG. 5G shows an example nonuniform stitching pattern 583 in which a filament 584 forms a series of dots within the substrate, thereby forming a mattress-like surface to the implant. This stitching pattern may be useful in situations where it is desirable to minimize the amount of surface area of the implant that the filament 584 takes. The dots may be equidistantly distributed or non-equidistantly distributed on the substrate surface. The dots may be randomly (or pseudo-randomly) distributed on the substrate surface, or distributed in a predetermined pattern on the substrate surface. The dots may be the same size or be different sizes.

FIG. 5H shows an example nonuniform stitching pattern 593 in which a filament 594 forms a series of disconnected curved lines within the substrate. The disconnected curved lines may be equidistantly distributed or non-equidistantly distributed on the substrate surface. The disconnected curved lines may be randomly (or pseudo-randomly) distributed on the substrate surface, or distributed in a predetermined pattern on the substrate surface. The disconnected curved lines may be the same size (e.g., length) or be different sizes (e.g., lengths).

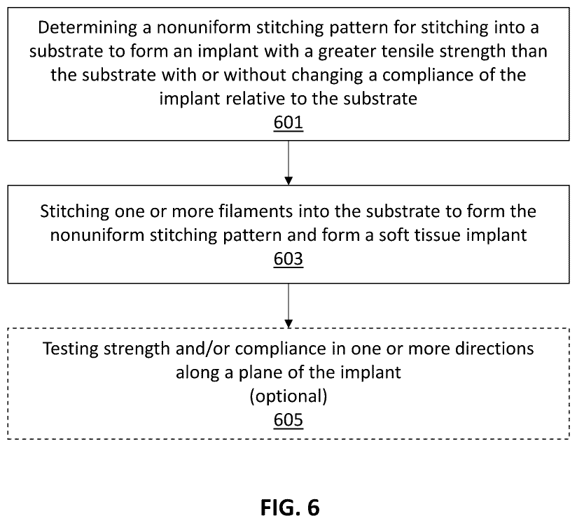

FIG. 6 is a flowchart indicating an example method of forming an implant having a nonuniform stitching pattern. At 601, a nonuniform stitching pattern is determined for stitching into a substrate to form an implant with a greater local tensile strength than the substrate. As discussed, the nonuniform stitching pattern may not change (or not substantially change) an overall compliance of the implant relative to the substrate, or makes the implant more compliant in a particular direction in the plane of the substrate. The nonuniform stitching pattern may be chosen based on desired strength and compliance characteristics of the implant.

At 603, one or more filaments are stitched into a substrate to form the nonuniform stitching pattern and form the soft tissue implant. If the substrate includes two or more layers of material, the filament(s) may be stitched into two or more of the layers. The substrate may be made of a biotextile or a medical textile, as described herein. The one or more filaments may be stitched in a lock-stitch configuration to form the nonuniform stitch pattern on a first side of the implant and a reverse stitch pattern on a second (opposite) side of the implant. The first side of the implant with the nonuniform stitch pattern may have a smoother surface contour and may include less filament surface area than the second side of the implant.

At 605, the implant is optionally tested for strength and/or compliance. In some examples, a uniaxial tensile testing procedure is performed, an example of which is described in U.S. Pat. No. 10,426,587, which is incorporated by reference in its entirety herein. The testing may be used to verify that a particular nonuniform stitching pattern imparts desirable tensile strength and compliance characteristics to an implant. Once verified, the nonuniform stitching pattern may be used in the manufacturer of soft tissue implants.

Once the implant is fabricated, the implant may be implanted into a patient's body in a particular orientation. For example, the first side of the implant with the nonuniform stitching pattern may be positioned nearer to one or more tissue types (e.g., organs) compared to the opposing second side with the reverse stitching pattern. Once implanted, the substrate and the embroidered filament can bear the load of tensile forces applied by the patient's tissues and the implant can comply as needed.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached, or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

The terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components, or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An implant for repairing or reconstructing soft tissue, the implant comprising:
a substrate comprising a biotextile or a medical textile having an upper surface and a lower surface; and
a nonuniform stitching pattern embroidered into the substrate that increases a strength of the implant relative to the substrate without changing a compliance of implant relative to the substrate, wherein the nonuniform stitching pattern comprises one or more filaments stitched into curved lines forming a plurality of adjacent cells having non-parallel sides across the upper surface of the substrate.

2. The implant of claim 1, wherein the one or more filaments stitched into curved lines has a radius of curvature that varies along the curved lines.

3. The implant of claim 2, wherein an absolute value of the radius of curvature is greater than 3 millimeters.

4. The implant of claim 1, wherein the nonuniform stitching pattern is configured so that the one or more filaments intersect at corners of the plurality of adjacent cells at a shared intersection stitching point.

5. The implant of claim 1, wherein the plurality of adjacent cells have a plurality of different shapes comprising two-sided, three-sided, four-sided, five-sided, and six-sided shapes.

6. The implant of claim 1, wherein the plurality of adjacent cells form an irregular pattern across the upper surface of the substrate.

7. The implant of claim 1, wherein the substrate comprises a plurality of layers of material.

8. The implant of claim 7, wherein the nonuniform stitching pattern is stitched into two or more of the plurality of layers of materials.

9. The implant of claim 1, wherein the substrate comprises a sheet of extracellular matrix material (ECM).

10. The implant of claim 1, wherein the implant is configured as a graft for one or more of hernia repair and tissue reconstruction.

11. The implant of claim 1, wherein the plurality of adjacent cells have a distribution of surface areas greater than 1 mm².

12. The implant of claim 1, wherein the plurality of adjacent cells have a distribution of areas between about 1 mm² and 100 mm².

13. The implant of claim 1, wherein the plurality of adjacent cells have approximately the same area.

14. The implant of claim 1, further comprising a border stitch around a perimeter of the implant.

15. The implant of claim 1, wherein the one or more filaments comprises a biocompatible material.

16. The implant of claim 15, wherein the biocompatible material is resorbable.

17. An implant for repairing or reconstructing soft tissue, the implant comprising:
a substrate comprising a biotextile or a medical textile having an upper surface and a lower surface; and
a nonuniform stitching pattern embroidered into the substrate, wherein the nonuniform stitching pattern comprises one or more filaments stitched into curved lines forming a plurality of adjacent cells having non-parallel sides across the upper surface of the substrate, wherein the one or more filaments stitched into curved lines has a radius of curvature that varies along the curved lines, so that the implant has the same compliance in any direction in the plane of the substrate.

18. A method for forming an implant for repairing or reconstructing soft tissue, the method comprising:
stitching one or more filaments into a substrate to form a nonuniform stitching pattern in the substrate, the substrate comprising a biotextile or a medical textile, where the nonuniform stitching pattern increases a strength of the implant relative to the substrate without changing a compliance of implant relative to the substrate, wherein the nonuniform stitching pattern comprises the one or more filaments stitched into curved lines forming a plurality of adjacent cells having non-parallel sides across an upper surface of the substrate, so that the implant has the same compliance in any direction in the plane of the substrate.

19. The method of claim 18, further comprising testing the compliance in one or more directions along the plane of the substrate to confirm that the implant has the same compliance in any direction in the plane of the substrate.

20. The method of claim 18, further comprising determining the nonuniform stitching pattern.

21. The method of claim 18, wherein the substrate includes one or more layers of the biotextile or the medical textile, wherein stitching the one or more filaments into the substrate comprises stitching the one or more filaments into the one or more layers of the biotextile or the medical textile.

22. The method of claim 18, wherein stitching the one or more filaments into the substrate comprises stitching the one or more filaments so that the curved lines have radius of curvature that varies along the curved lines.

23. The method of claim 18, wherein stitching the one or more filaments into the substrate comprises stitching the one or more filaments in a lock-stitch configuration.

24. The method of claim 18, wherein stitching the one or more filaments into the substrate comprises stitching one or more non-parallel lines, curved shapes, circles, ovals, irregular shapes, loops, dots, or any combination thereof into the substrate.

25. A method of using an implant for repairing or reconstructing soft tissue, the method comprising:
surgically implanting the implant with a patient's body, wherein the implant includes a nonuniform stitching pattern embroidered into a substrate that increases a strength of the implant relative to the substrate without changing a compliance of implant relative to the substrate, wherein the nonuniform stitching pattern comprises one or more filaments stitched into curved lines forming a plurality of adjacent cells having non-parallel sides across an upper surface of the substrate.

26. The method of claim 25, wherein surgically implanting the implant comprises orienting the upper surface of the substrate nearer to a particular tissue type of the patient's body relative to a lower surface of the substrate that is opposite the upper surface.
